(19) United States
(12) Patent Application Publication
(10) Pub. No.: US 2025/0198956 A1
(43) Pub. Date: Jun. 19, 2025

(54) AUTOMATIC SYSTEM AND METHOD FOR DETERMINING AN OXIDATION LEVEL IN A FOOD SAMPLE

(71) Applicants: Zeev WIESMAN, Lehavim (IL); Charles LINDER; Tatiana OSHETER, Ashdod (IL); Salvatore (Moshe-David) CAMPISI-PINTO, Haifa (IL)

(72) Inventors: Zeev WIESMAN, Lehavim (IL); Charles LINDER; Tatiana OSHETER, Ashdod (IL); Salvatore (Moshe-David) CAMPISI-PINTO, Haifa (IL)

(21) Appl. No.: 18/846,475

(22) PCT Filed: Mar. 12, 2023
(86) PCT No.: PCT/IL2023/050254

§ 371 Date: Sep. 12, 2024

Related U.S. Application Data
(60) Provisional application No. 63/319,334, filed on Mar. 13, 2022.
(60) Provisional application No. 63/400,085, filed on Aug. 23, 2022.
(60) Provisional application No. 63/443,454, filed on Feb. 6, 2023.

(51) Int. Cl. G01N 24/08; G01N 33/02; G06N 20/00

(52) U.S. Cl. CPC G01N 24/08; G01N 33/02; G06N 20/00

(57) ABSTRACT

A method for determining oxidation level in a sample, comprising: (A) a training stage comprising: (A.1) providing a plurality of food samples; submitting each food sample to an LF-H1-NMR device and extracting NMR data for that sample; (A.2) determining in a lab an oxidation level of each sample; (A.3) storing for each said samples a record reflecting the extracted NMR data and a respective oxidation level; (A.4) repeating steps A.1 to A.4 for all samples; (A.5) given said plurality of sample records, training and creating a machine-learning unit that, given a sample's NMR data at the unit's input, indicates an oxidation level; and (B) a real-time stage comprising: (B.1) during real-time, extracting real-time NMR data for a food sample; (B.2) submitting the real-time NMR data to said machine-learning unit; and (B.3) based on said real-time data, determining by said machine learning unit a respective oxidation level for that sample.

### FIELD OF THE INVENTION

The invention generally relates to systems and methods for measuring and determining food quality. More particularly, the invention relates to an artificial intelligence (AI) NMR-based autonomic system and method for determining and profiling an oxidation level in food ingredients.

### BACKGROUND OF THE INVENTION

Organic materials' oxidation levels are typically measured using tedious, costly, and time-consuming lab methods. These measurements require chemical lab facilities and are labor intensive too.

Low field (LF) nuclear magnetic proton (¹H) resonance (NMR)—(¹H NMR, NMR, and LF H¹ NMR and LF-NMR are terminologies that are the same connotation in this patent application) is a spectroscopic technique used to elucidate the chemical and physical/morphological structure of organic compounds and to monitor reactions they undergo with chemical and morphological changes, and porous structures of water-containing inorganics and ceramics.

Multi-dimensional maps typically present data collected by NMR measurements. Such maps provide an easy-to-understand format. For example, U.S. Pat. No. 7,388,374 describes interpretation methods for NMR maps based on measurements taken on a fluid sample from a borehole.

Data acquired by NMR measurements include proton (¹H) spin-lattice/matrix energy relaxation time (T₁) and spin-spin energy relaxation time (T₂). Material relaxation processes permit the population of H¹ nuclear spins to return to equilibrium after the absorption of radio frequency energy delivered by the NMR instrument, either through a mechanism of spin-lattice interactions (the lattice/matrix is the environment around the ¹H nucleus, namely, neighboring atoms or molecules) or the mechanism of spin-spin interactions. T₁ and T₂ are the time constants associated with the energy equilibrium value. T₁ indicates how fast the magnetization relaxes back along the z-axis by spin-lattice interactions (so it is called longitudinal relaxation time), and T₂ measures how fast the spins exchange energy in the transverse (x-y) plane spin-spin interactions (so it is called transverse relaxation time).

One-dimensional analysis of either spin-lattice or spin-spin energy relaxation times, that is, conversion of low-field NMR relaxation signals into a continuous distribution of either T₁ or T₂, resulting in a graph of different peaks as a function of T₁ or T₂, was demonstrated in the literature. In addition, uniform penalty inversion of two-dimensional NMR relaxation data based on Tikhonov-like regularization was also reported.

The efficient rapid monitoring of food quality and safety during the entire food cycle, including from harvesting/breeding, transportation, storage, pre-preparation, cooking, to the final step of digestion, has not yet been achieved and is a fundamental issue in foods that contain carbon-carbon double bonds such as in polyunsaturated alkyl chains. These are found in many foods susceptible to deterioration because of oxidation into toxic products. Current methodologies are not on-line-efficient and not readily applicable in applications, such as but not limited to online analysis for food product production, examples of components susceptible to oxidation at different stages of production storage, transportation, cooking, and in some embodiments during digestion as in the acidic conditions of the stomach. The term “online” analysis refers herein to analysis results provided at most within several minutes.

U.S. Pat. No. 11,189,363 partially by the same inventors as of the present application discloses time-domain nuclear magnetic resonance (TD NMR) technologies for monitoring oxidative changes in a variety of food susceptible to oxidation, such as many different seeds, oils, emulsions, vegetables, and fish and meat products. While these technologies provide significant improvements, they are still inefficient, not cost-effective, and too slow to completely satisfy the demand for online analysis of food oxidation, especially for food components that are highly susceptible to oxidation.

Moreover, U.S. Pat. No. 11,189,363 discloses a method for characterizing chemical and/or morphological features of a material, comprising acquiring relaxation data from LF ¹H NMR (Low-Field Nuclear Magnetic Resonance) measurements of the material, converting the relaxation signals into a multidimensional distribution of longitudinal and transverse relaxation times by solving an inverse problem under both L1 and L2 regularizations, and further imposing a non-negativity constrain. The respective regularization parameters λ1 and λ2 control the amount of regularization applied to the model. They are selected using cross-validation, and are set based on the signal-to-noise level of the measurements, the signal intensity, the dimensions of the acquired set of data, and the dimensions of an input matrix of distribution components of interest. One or more characteristics of the material are identified with the aid of the multidimensional T₁-T₂ distribution, for example, the position and intensity of peaks on T₁ vs. T₂ spectrums and the T₁/T₂ ratios as described below. The T₁ is a spin-matrix relaxation time and the T₂ is spin-spin relaxation time. This spectrum generation consumes typically from several to tens of minutes to hours. The identification of the chemical and morphological structures assigned to individual peaks of the T₁ vs. T₂ spectrum requires extensive material analysis by High Field NMR, X-ray, microscopy, FTIR (Fourier Transform Infrared spectroscopy) and MS (Mass Spectrometry) and material substitution. Once assigned the peak assignment can be used repeatedly for other LF ¹H NMR spectrums of the same material categories.

Also relevant to the present invention is a recently published article by the inventors of the present invention: “Alkyl Tail Segments Mobility as a Marker for Omega-3 Polyunsaturated Fatty Acid-Rich Linseed Oil Oxidative Aging”, September 2020, Journal of the American Oil Chemists' Society 97(12). This article discloses a sensorial LF ¹H NMR energy relaxation time application based on monitoring primary chemical and structural changes occurring with time and temperature during oxidative thermal stress for better and rapid evaluation of LSO's (Linseed Oil) aging process. The article also discloses the rapid characterization of materials undergoing oxidation and the identification of material composition; the emphasis is on foods to minimize the oxidation of components susceptible to oxidation. The study disclosed therein, however, does not measure T₁ (spin-lattice) relaxation times but focuses on different T₂ times of energy relaxations due to spin-spin coupling, and in this way, proton motion/mobility of linseed oil (LSO) molecular segments are monitored to characterize the chemical and structural changes in all phases of the autoxidation aging process. This work showed that LSO tail segment mobility in terms of T₂ multi-exponential energy relaxation time decays, generated by data reconstruction of ¹H transverse relaxation components, provide a relatively rapid, clear, sharp, and informative understanding of LSO sample's autoxidation aging processes.

The prior art techniques described above (such as tail T2 time-domain monitoring) are inefficient for online and rapid analysis of food products' toxic contents with an emphasis, but not limited to, oxidized products found in foods with di or polyunsaturated fatty acids (such as seeds, oils, emulsions, and fish and meat) that are generated during cooking, such as but not limited to, frying in oils of French fries. This alkyl tail analysis is, however, relatively rapid in comparison, for example, to the analysis of a food's chemical and morphological state as described by T1 and T2 measurements that requires many minutes, far too long for efficient rapid food quality control with an emphasis on oxidized products found in foods with components with multiple double bonds.

It is an object of the invention to provide a system for performing a fast, efficient, and reliable determination of the oxidation level in food products.

Another object of the invention is to provide this system in compact size and with the capability of fast-online performance.

Other advantages of the invention become apparent as the description proceeds.

### SUMMARY OF THE INVENTION

The invention relates to a method for determining a level of oxidation in a sample, comprising: (A) a training stage comprising: (A.1) providing a plurality of food samples; submitting each said food sample to an LF-H¹-NMR device and extracting NMR data for that sample; (A.2) determining in a lab an oxidation level of each one of said samples; (A.3) storing in a database for each one of said samples a record reflecting the extracted NMR data and a respective oxidation level; (A.4) repeating steps a-d for all said plurality of samples; (A.5) given said plurality of sample records in the database, training and creating a machine-learning unit that, given a sample's NMR data at the unit's input, determines and indicates an oxidation level at the unit's output; (B) a real-time stage comprising: (B.1) during real-time, extracting real-time NMR data for a food sample; (B.2) submitting the real-time NMR data to said machine-learning unit; and (B.3) based on said real-time data, determining by said machine learning unit a respective oxidation level for that sample.

In an embodiment of the invention, the sample is a food sample containing oxidation-susceptible components.

In an embodiment of the invention, the NMR data is selected from one or more of, NMR T1 energy relaxometry data, NMR T2 relaxometry data, and NMR T1-T2 energy relaxometry data.

In an embodiment of the invention, each said record forms labeled data for use at the training stage of the machine learning unit.

In an embodiment of the invention, each said oxidation level is reflected by relaxometry and self-diffusion signals acquired from the sample.

In an embodiment of the invention, the NMR data comprising exponential decay curves.

In an embodiment of the invention, the machine learning training and operation are based on pattern recognition of crude proton energy-time decay curves.

In an embodiment of the invention, the real-time stage is performed online during one or more of the food's preparation, storage, transportation, or cooking phases.

In an embodiment of the invention, the sample being analyzed for oxidation contains mono or polyunsaturated fatty acids (PUFA), either in solid, liquid, or emulsion combining different phases.

The invention also relates to a system for determining a level of oxidation in a sample, comprising: (a) an LF-NMR device configured to extract NMR data from a sample and convey the same into a pre-trained machine-learning unit; and (b) a pre-trained machine-learning unit configured to receive said NMR data and to determine a level of oxidation within said sample based on said NMR data.

In an embodiment of the invention, the sample is a food sample containing oxidation-susceptible components.

In an embodiment of the invention, the NMR data is selected from one or more of, NMR T1 relaxometry data, NMR T2 relaxometry data, and NMR T1-T2 relaxometry data.

In an embodiment of the invention, each said oxidation level is reflected by relaxometry and self-diffusion signals acquired from the sample.

In an embodiment of the invention, the determination of the oxidation level is based on pattern recognition of crude proton energy decay curves.

In an embodiment of the invention, the system is configured for online determination of the oxidation level during one or more of the food's preparation, storage, transportation, or cooking phases.

### BRIEF DESCRIPTION OF THE DRAWINGS

In the drawings:

FIG. 1 illustrates in block diagram form the general structure of a system for determining the oxidation level in a food (or another organic) sample, according to an embodiment of the invention;

FIG. 2 shows key elements of the system of the invention;

FIG. 3 is a flow diagram illustrating the training process according to an embodiment of the invention;

FIG. 4 illustrates how the present invention applies machine-learning techniques to classify the oxidation level of oil samples;

FIG. 5 schematically summarizes the system's setup and the typical workflow for analyzing oil samples;

FIG. 6 shows an example of a tailor-made Convolutional Neural Network (CNN) architecture used in the invention;

FIG. 7 shows how diffusion coefficients correlated with various oxidation measurements in an experiment;

FIG. 8 provides test results showing the accuracy of diffusion coefficients as an estimator of the oxidative treatment imposed on oil samples;

FIG. 9 provides test results showing the accuracy of total oxidation as an estimator of the oxidative treatment imposed on the oil samples;

FIG. 10 shows raw exponential decay curves of 1H NMR T2 relaxation signals of linseed oil collected by CPMG method from LF NMR after different thermal oxidation levels/times; and

FIG. 11 illustrates accuracy and loss functions for 30 different Convolutional Neural Networks (CNNs) training sessions.

### DETAILED DESCRIPTION OF PREFERRED EMBODIMENTS

The invention relates to a system and method for determining food oil oxidation levels using LF ¹H NMR signatures and pattern recognition (PR) machine learning (ML) techniques. For example, the system can determine oxidation levels in food and its ingredients in an automated, fast, and affordable manner. In addition, the invention may also utilize self-diffusion sample values (D or SD, equivalent in this application) as measured by LF ¹H NMR as a rapid indicator of material oxidation.

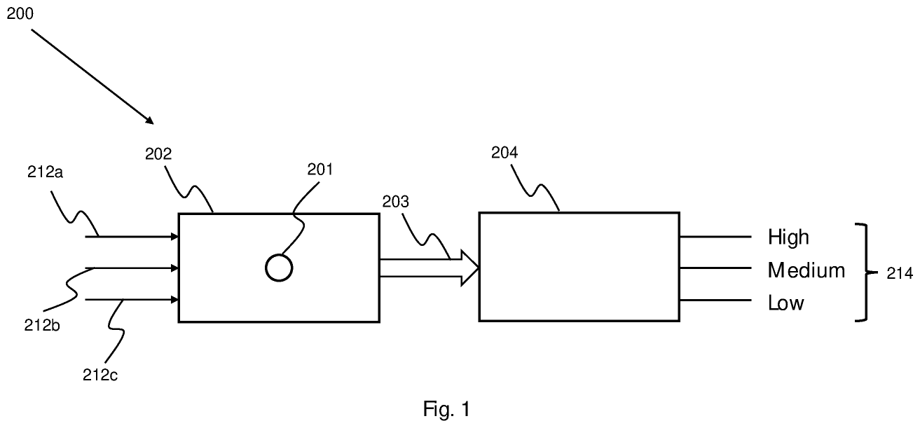

FIG. 1 illustrates in block diagram form the general structure of a system 200 for determining the oxidation level in a food (or another organic) sample, according to an embodiment of the invention. The system generally includes a conventional NMR apparatus (or machine) 202 configured to apply a conventional TD-NMR test on (typically food) sample 201. The test may include one or more relaxation procedures 212, such as a proton (¹H) spin-lattice/matrix energy relaxation time (T₁) 212a, spin-spin energy relaxation time (T₂) 212b, or a combination thereof 212c. The NMR relaxation test result is a vector 203 (in matrix or visual-graph format) defining the time domain (TD) relaxation response of sample 201 to one or more of the procedures 212. Next, the TD response vector 203 is conveyed to a pre-trained machine learning (AI) analyzer 204. Finally, the pre-trained machine learning analyzer 204 classifies the input vector into a most suitable class of oxidation level. For example, in one embodiment, analyzer 204 classifies the NMR output vector 203 into high or low oxidation levels 214. In another embodiment, analyzer 204 classifies the NMR output vector 203 into high, medium, or low oxidation levels 214. More classes may be included, depending on the apparatus's structure and training resolution.

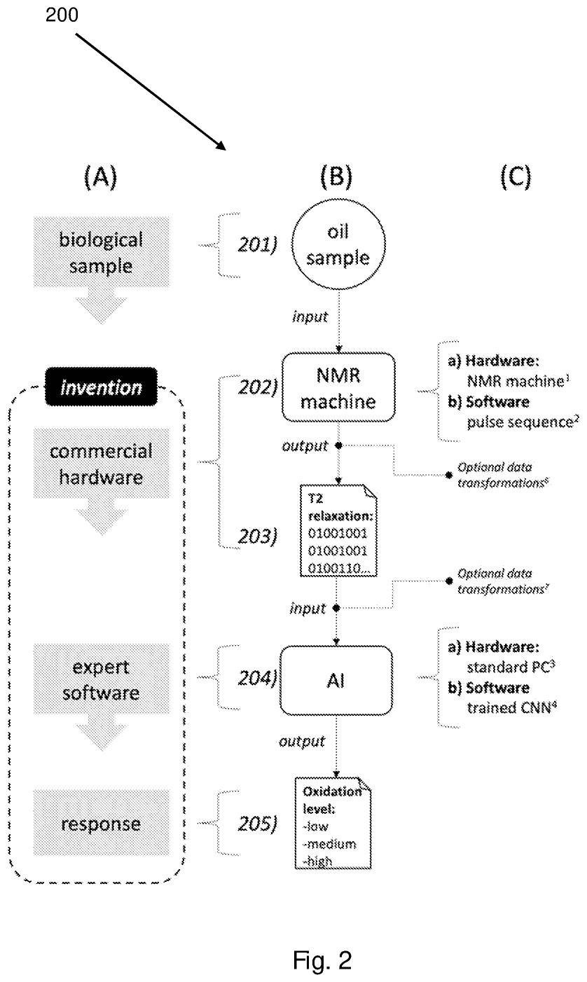

FIG. 2 shows key elements of the system of the invention. Columns A, B, and C indicate various specifications. The invention combines a Low Field Nuclear Magnetic Resonance (NMR) machine and a Convolutional Neural Network (CNN). The NMR machine investigates a food sample, generating a T2 relaxation curve—a complicated signal that cannot be understood directly. The CNN interprets the T2 relaxation curve and provides information on the oil sample's oxidation level. Following the one-time creation of the CNN, the process is simple, efficient, and can be performed online. More specifically, FIG. 2 generally illustrates the structure of system 200 in more detail. An organic sample (for example, oil or another food) 201 is positioned within the NMR machine 202. NMR machine 202 includes an inexpensive processing unit, such as a desktop, that scans the organic sample 201 utilizing one or more procedures 212 (FIG. 1). Procedures 212 are one or more of T₁, T₂, or T₁-T₂ energy relaxation times. The scan results in a response vector 203 associated with the organic sample. The software operating the NMR may be any conventional NMR software. The system combines a Low Field Nuclear Magnetic Resonance (NMR) machine 202 and a machine learning (AI) unit 204, preferably a convolutional Neural Network (CNN). For example, the pre-trained NMR machine processes a food sample and generates a T2 relaxation curve—a complicated signal that cannot be interpreted directly. NMR 202 may provide the T2 response as a relaxation curve, possessing information on the sample's oxidation level. The process is simple and efficient.

The NMR report 203 may have the form of a vector of numeric values, such as a matrix. Alternatively, the report may have a relatively more user-friendly (transformed) output, like a graphical plot fabricated for an interpretation of a specialized (highly trained) operator. The NMR's output is fed to an expert AI (204). AI 204 is a machine-learning apparatus previously trained to recognize oxidation patterns given the NMR output vector. The AI training process is described below. The final result (205) is a simple classification indicating the oxidation level within the biological sample 201.

For example, vector 203 may represent the inverse Laplace transform (ILT) spin-lattice (T₁) and spin-spin (T₂) energy relaxation signature graphs of thermally oxidized oil, while machine learning apparatus 204 correlates the same (together with a self-diffusion coefficient (D)), to chemical and morphological changes in the oil.

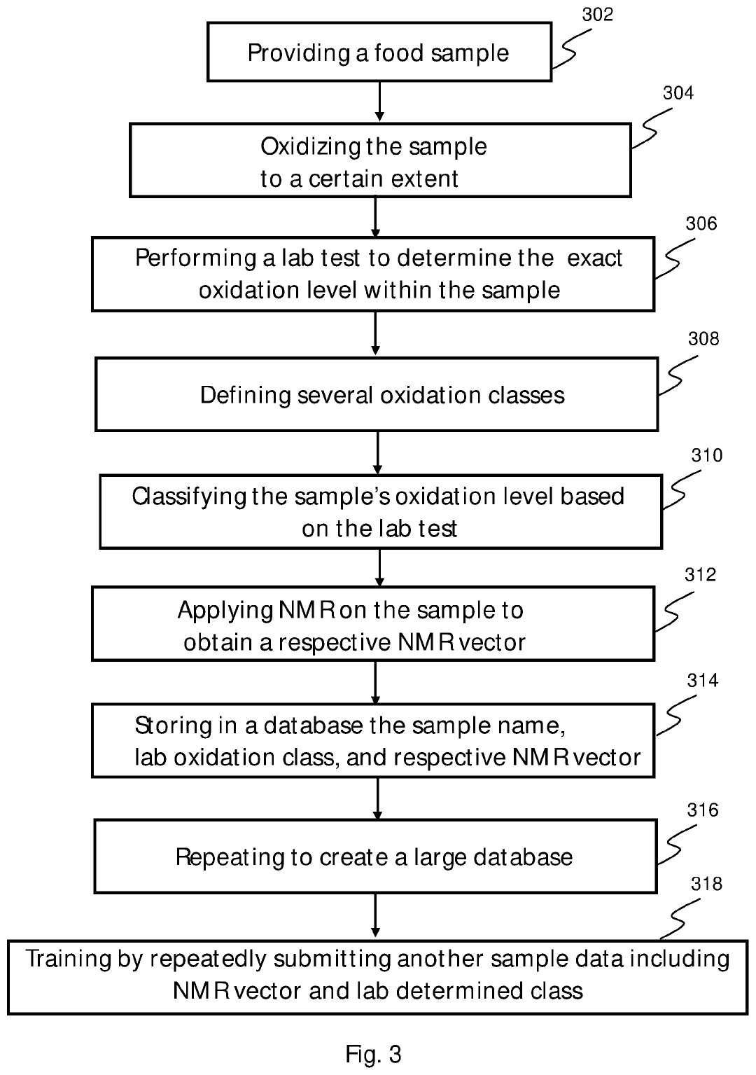

FIG. 3 is a flow diagram illustrating the training process according to an embodiment of the invention. In step 302, a first food sample is selected. In step 304, the sample is oxidized to a certain brief level. In step 306, a lab test is performed on the sample to verify the oxidation level. Next, in step 308 several oxidation levels are defined, such as (a) a two-class space, such as “low” and “high”, or (b) a three-class space including “low,” “medium”, and high” oxidation levels. In step 310, the sample's oxidation level, as determined in the lab, is classified based on the classes defined in step 308. Next, in step 312 an NMR test is applied to the sample, resulting in a respective NMR vector. Next, steps 302 to 314 are repeated with many samples to create a large database, including hundreds or thousands of samples and their respective data (as indicated). Finally, in step 318, the training of the machine-learning unit is performed by sequentially submitting to the machine those classified samples from the database until convergence. As detailed below, the inventors have designated a portion of the database to the training while keeping the remaining samples to verify the system's applicability and accuracy.

### Further Discussion and Experiments

The invention utilizes LF ¹H NMR signatures and machine learning (ML) techniques for pattern recognition (PR) of oxidation levels in food (such as oil). The oxidation level in food and food ingredients is determined automatically, quickly, and affordably. The invention utilizes a sample's self-diffusion (D or SD, equivalent in this application) values, measured by an NMR machine, as a rapid indicator of material oxidation.

For example, the invention demonstrates the ability to measure the inverse Laplace transform (ILT) spin-lattice (T₁) and spin-spin (T₂) energy relaxation signature graphs of thermally oxidized oils and the correlation of these values with the self-diffusion coefficient (D), reflecting chemical and morphological changes in the oil. The relationship between the D value of each sample was calculated from its T₂ values To reduce the time required for the NMR sensor to characterize oil quality and its degree of oxidation. At the same time, the thermal and air conditions enhancing oil oxidation were also formulated by combining radio-frequency pulses, as mentioned above. A high (R²>0.90) rate of correlation between D and oil oxidation's conventional colorimeter standard tests (e.g., PV, p-Anisidine, and TOTOX) was experimentally demonstrated. The results were verified in a high-temperature (80° C.) oxidation study of saturated, monounsaturated, and polyunsaturated edible oils, such as butter, coconut, olive, canola, soy, and linseed. Furthermore, cluster analysis clearly showed that self-diffusion D values, reflecting the average mobility of the sample's ¹H protons, is an excellent rapid (<1 minute) marker/indicator for the oil's quality (with emphasis on the oil's oxidation status). Therefore, the system of the invention can accurately be used in the oil industry to measure oxidation levels.

The inventors also established that rapid online monitoring of single and multiple-phase emulsion oil oxidative modifications is described using T₂ generation of samples' average self-diffusion (SD) values and their changes upon oxidation. SD values of the entire sample are rapidly determined on intact samples. They can readily characterize and quantify the different stages of fatty acid (FA) oil or oil in water emulsion oxidation and the extent of oxidation. The SD values correlate well with changes in aldehyde and peroxide and total oxidation (TOTOX) values. The procedure of the invention for the oxidation level determination can be carried out within one to several minutes on intact unmodified samples, compared to many minutes to several hours needed by other methodologies. In one embodiment, The SD values are rapidly determined on intact samples—they can be used to readily characterize the different stages of fatty acid (FA) oil or oil in water emulsions oxidation and the extent of oxidation.

The approach of the present invention has been developed based on:
i. A large database included the results of multiple types of analysis of oil-rich food products tested with LF TD NMR relaxometry, among others (a) TD NMR relaxation spectral T₁-T₂ fingerprints; (b) self-diffusion material values; (c) crude proton decay curves; and (d) standard measures of oil oxidation. The above data was used to create the machine learning (ML) unit 204.
ii. A machine learning (ML) algorithm based on a proprietary database (as in i).
iii. An optimization training software for creating an operational machine-learning unit capable of identifying oxidation level in a sample. The operational machine-learning unit is based on (i) and (ii) above.

The system (i, ii, iii) provides solutions where the human-based mechanistic formula cannot handle the complexity of the generated data.

### Training, Testing, and Validating the AI of the Present Invention

The invention's system utilizes machine learning algorithms to learn from data rather than relying on formulas based on physics and chemistry. The system classifies various complex arrangements of chemical and morphological features in foods, including fatty acid emulsions and micelles, eggs, oils, surface chemistry, and the structure of foods cooked in oil, like French fries, chicken, pancakes, etc. The machine-learning unit of the invention was trained using hundreds of NMR experiments that generated T1 and T2 relaxation and self-diffusion signals. The experiments were augmented with data on the oxidation level measured by standard methods like peroxide value (PV), p-anisidine (PAV), and TOTOX. The techniques were tested and validated with this large collection of NMR data.

Generally, there are three major ML types: i) supervised, ii) unsupervised, and iii) reinforcement ML. As there is no systematic or obvious way to know “a-priori” the most efficient ML for a given dataset, the inventors tested different ML techniques and compared the results using available performance-evaluation metrics. The inventors applied supervised learning (learning from labeled data), as they developed a dataset including oil samples' measurements with labels. The dataset included the NMR readings and their corresponding oxidation levels for each food oil sample, as acquired by conventional chemical laboratory tests. The inventors also applied unsupervised learning to estimate the available variance within the NMR readings. The significant variance was welcome as it can carry reach features enabling distinction between fine scales of oxidation levels.

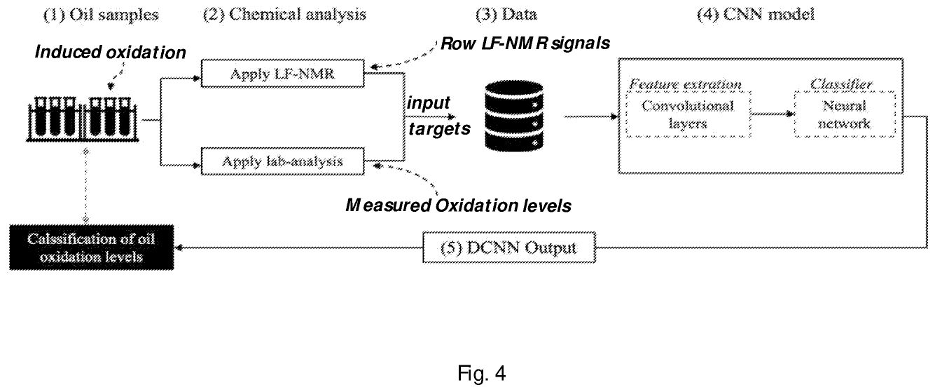

FIG. 4 illustrates how the present invention applies machine learning techniques to classify oil samples (1) according to their oxidation levels (5). For each oil sample, the technique includes: (2) LF NMR signal acquisition used as input in the CNN, and standard lab methods used as ground truth in the CNN. The data frame that combines signals and lab results, i.e. input and targets, is stored and used for the Convolutional Neural Network (CNN) learning process. In (4), the CNN consists of two major modules: feature extraction and classification. The classification results are provided at (5).

The core of the classification algorithm is an AI (ANN) module 204 (FIG. 2) trained to recognize oxidation levels based on NMR data files. During training, the AI learns to classify the oil based on the NMR data files (#3 in FIG. 4) using appropriate “learning material,” such as the chemometrics of the sample (the measured oxidation level). The training uses a combination of NMR data files and the oxidation level of the food oil, which serves as the supervisor for the machine learning algorithm (1, 2, and 3 in FIG. 4).

An untrained Artificial Neural Network (ANN) is a generic linear combiners (neurons) system that can be specialized for a task utilizing calibration. The task in this case is mapping NMR output files to an oxidation level. After training, the ANN accurately determines the oxidation level for a new, unseen NMR file. The training achieves generalization, meaning the ANN's ability to classify new NMR sequences into correct oxidation levels. In an embodiment of the invention, the ANN is based on a Convolution Neural Network (CNN), shown in FIGS. 3 and 4.

The end product of the invention is an autonomous system combining hardware (LF-NMR machine) and software (Convolutional Neural Network (CNN), or another type of ANN) to classify food sample's oxidation level into three (or more) categories based on the sample's NMR signal. In brief, the novelty of the system resides in: i) the unique data workflow and ii) the optimized machine learning configuration to improve classification accuracy.

The Convolutional Neural Network (CNN) is a machine learning algorithm used for multinomial classification involving classifying instances into multiple different classes. The CNN combines a process of automatic feature extraction and supervised learning through artificial neural networks to classify the signals effectively. It is a form of pattern recognition and the process of adapting a pre-existing model to a specific problem is called fine-tuning or learning. After the learning phase is completed, the trained CNN can be deployed to classify the oil's oxidation level based on T2 signals.

One specific embodiment of the system of the invention includes a 1D Convolutional Neural Network (CNN) with four convolutional layers and two dense layers as follows:
1. Input layer: a first layer that receives the input data, in this case, a 1D array representing the NMR signal.
2. Convolutional Layers: Layers that perform convolution operations on the input data, typically using a set of learnable filters. The network has four convolutional layers, each applying multiple filters to extract features from the input data.
3. ReLU activation: A typical activation function that is applied after each convolutional layer to introduce non-linearity into the network. The ReLU activation replaces all negative values in the output of a convolutional layer with zeros.
4. Max Pooling: This is a down-sampling operation applied after some of the convolutional layers to reduce the output data size and computation complexity.
5. Flattening layer: After the final convolutional layer, the output is transformed from a multi-dimensional array to a 1D array, which is then passed to the dense layers.
6. Dense layers: These are fully connected layers that make predictions based on the input data. In this case, the network has two dense layers, each performing a matrix multiplication between the input and a set of learnable weights, followed by an activation function.
7. Output layer: This is the final layer of the network that provides the prediction based on the input data, in this case, the oxidation level of the oil sample.

In another feasible setup, the Convolutional Neural Network (CNN) processes data obtained from the LF-NMR sensor. The data undergoes processing to form relaxation curves and is then transformed through an inverse Laplace transformation (ILT), resulting in graphic spectra. These two steps provide insightful chemical and structural information but take longer for signal collection (especially T₁) and ILT processing.

The system of the invention meets the oil and food industry requirement for a rapid online evaluation of oil oxidation levels. For this purpose, it is sufficient to use only data from LF-NMR T₂ raw relaxation signals curve rapidly collected from the magnetic field of the NMR. The capability of the computing system to differentiate between different relaxation curves is significantly higher than human capability. Therefore, using the relatively fast extraction of basic relaxation curves (e.g., a few seconds) is sufficient to differentiate and classify the oxidation status of the tested oils. Furthermore, to gather more highly relevant information from the LF-NMR and to increase confidence in the classification of the oils, fast self-diffusion coefficient D data collected by a gradient pulse in the LF-NMR sensor from each tested oil sample is also used. These two collected LF-NMR relaxation parameters have been found well correlated with conventional lab chemical standard tests of oils oxidation (PV, p-AV and TOTOX). Therefore, the automated process and system significantly simplify the determination of oxidation levels and are well-suited for industrial applications as they provide analysis faster by several orders than conventional techniques.

In the following examples, the self-diffusion (D or SD are interchangeable), measurements were carried out with a 20 MHz mini spec bench-top pulsed NMR analyzer (Bruker Analytic GmbH, Germany), equipped with a permanent magnet and a 10 mm temperature-controlled probe head. The self-diffusion coefficient D was determined by a pulsed-field gradient spin echo (PFGSE) method (Stejskal and Tanner, 1965). The pulse sequence was used with 16 scans, τ of 7.5 ms, and a recycle delay of 6 s. Typical gradient parameters were Δ of 7.5 ms, δ of 0.5 ms, time between the 90° pulse to the first gradient pulse of 1 ms, and G of 1.6T/m. Each reported self-diffusion coefficient (D) value is the average of ten measurements.

### Example 1

### Lr ¹HNMR Analytical Determination of Diffusion on Intact Samples VS. Total Oxidation

The inventors induced oxidation into PUFA containing oil samples using oxidative treatments of varying durations. Different treatments induce different oxidation levels. The resulting oxidation levels were measured using three different methods: i) peroxide value (PV)—standard colorimetric estimation of peroxide values and primary markers of oxidation, ii) p-anisidine (PAV)—consists of standard colorimetric estimation of secondary markers of oxidation, aldehydes, iii) self-diffusion (D)—consists of LF¹H-NMR analytical determination of diffusion property in the intact tested samples during oxidation process. The methods are complementary, as they reflect slightly different aspects of the chemical and morphological changes resulting from the treatment.

Annex 1 (see below) shows a sample of the available experimental measures. Annex 2 (below) shows the corresponding descriptive statistics.

Given these measurements, the inventors tested to which extent diffusion coefficients can predict (or explain) peroxide and anisidine measurements to characterize oxidative processes. In other words, to provide diffusion coefficients that can be rapidly measured on intact samples by LF ¹H-NMR: a) what can be inferred (predicted) about peroxide and anisidine measurements; and b) the error associated with those predictions.

Linear models (see Table 1 below) best explain the quantitative relationships between diffusion coefficients and the various oxidation measurements. For example, Model 1 shows that the diffusion coefficient can explain about 64% of the variance (R², R² adjusted) of Total-oxidation; moreover, for each 0.001 increment in diffusion, expect a decrease of −6.702 (95% CI: −7.729, −5.675) is expected in the corresponding total oxidation value. Overall, these models show that by measuring diffusion coefficients, the total oxidation and/or Peroxide-value and/or Anisidine-value can be inferred with medium/good accuracy (R², R² adjusted from 0.625 to 0.641). In practice, measuring Total oxidation (Peroxide-value+Anisidine-val.) or Peroxide-value only does not change the performance of the model.

TABLE 1
Three different models showing how diffusion coefficients
can predict oxidation levels measured in three different
ways (Total oxidation, Peroxide-value, Anisidine-value)

| | Model 1: Total-oxidation | | |
|---|---|---|---|
| Predictors | Estimates | CI | p |
| (Intercept) | 300.1 | 264.5 to 335.8 | <0.001 |
| Diffusion-coeff. | −6.7 | −7.7 to −5.6 | <0.001 |
| Observations | 96 | | |
| R2/R2 adjusted | 0.64/0.63 | | |
| | Model 2: Peroxide-val. | | |
| Predictors | Estimates | CI | p |
| (Intercept) | 300.18 | 264.5 to 335.8 | <0.001 |
| Diffusion-coeff. | −6.704 | −7.731 to −5.677 | <0.001 |
| Observations | 96 | | |
| R2/R2 adjusted | 0.64/0.63 | | |
| | Model 3: Anisidine-val. | | |
| Predictors | Estimates | CI | p |
| (Intercept) | 210 | 184.9 to 235.1 | <0.001 |
| Diffusion-coeff. | −4.55 | −5.2 to −3.8 | <0.001 |
| Observations | 96 | | |
| R2/R2 adjusted | 0.62/0.62 | | |

CI is the 95% confined interval
P is the p-value.

R² is the proportion of the variance in the dependent variable that is predictable from the independent variables. It is a value between 0 and 1, where a value of 1 means that the model perfectly fits the data and all the variability in the response is explained by the independent variables. A value close to 0 indicates that the model doesn't fit the data well, and only a small proportion of the variability in the response is explained by the independent variables.

R² adjusted is a modified version of the R-squared statistic in regression analysis, which adjusts for the number of predictors in the model. The adjusted R-squared considers the number of independent variables and the sample size, and provides a more accurate measure of the model's goodness-of-fit by penalizing the addition of variables that do not improve the model's performance. A higher adjusted R-squared value indicates a better fit of the model to the data than a model with a lower adjusted R-squared value.

Results in Table 2 show that 80% of the residual errors are between −53.68 and +54.31 total oxidation units (TOU); conversely, 20% of predictions will have a larger error, up to the extreme between −85.31 and 179.75. Similarly, 50% of prediction will be associated with an error between −30.51 and 26.10 (TOU).

TABLE 2
Residual errors of Model 1 (in terms of Total
oxidation value)

| Quantile | 0% | 10% | 20% | 30% | 40% | 50% | 60% | 70% | 80% | 90% | 100% |
|---|---|---|---|---|---|---|---|---|---|---|---|
| Total-oxidation | −85.31 | −53.68 | −38.46 | −30.51 | −22.06 | −2.64 | 5.76 | 26.1 | 37.71 | 54.31 | 179.75 |
| residual error | | | | | | | | | | | |

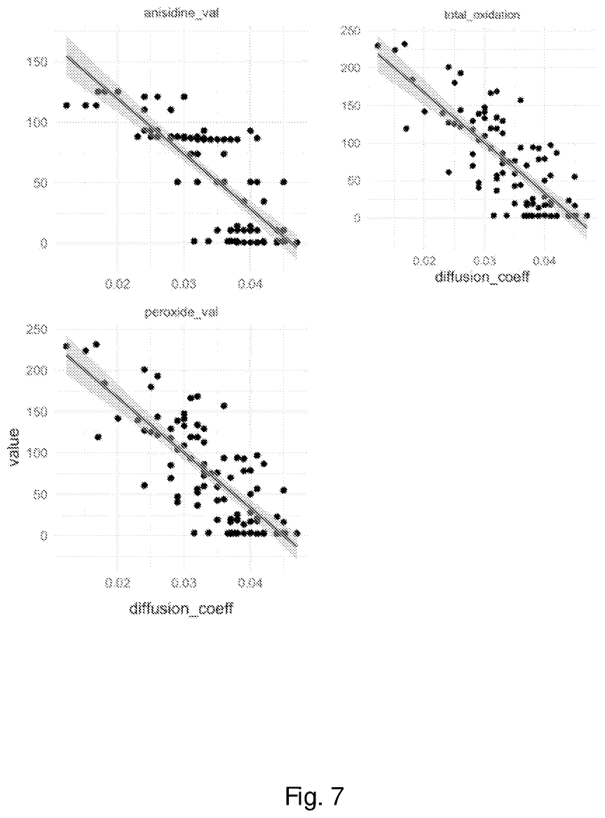

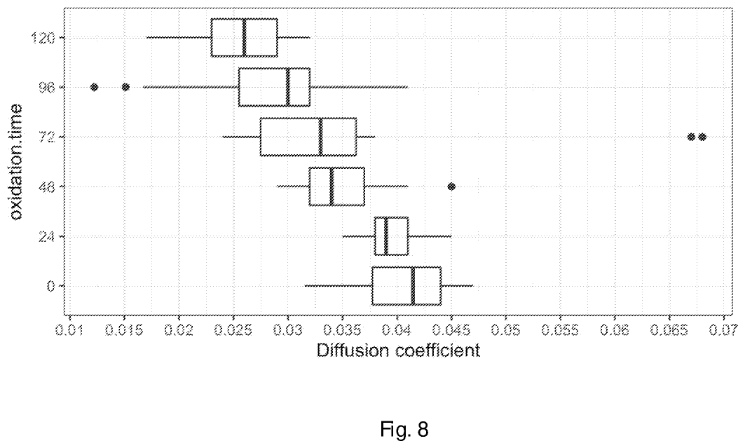

FIG. 7 shows how diffusion coefficients correlate with the various oxidation measurements. FIG. 8 shows the distribution of the magnitude of the error committed when estimating oxidation values utilizing linear regressions of diffusion coefficients.

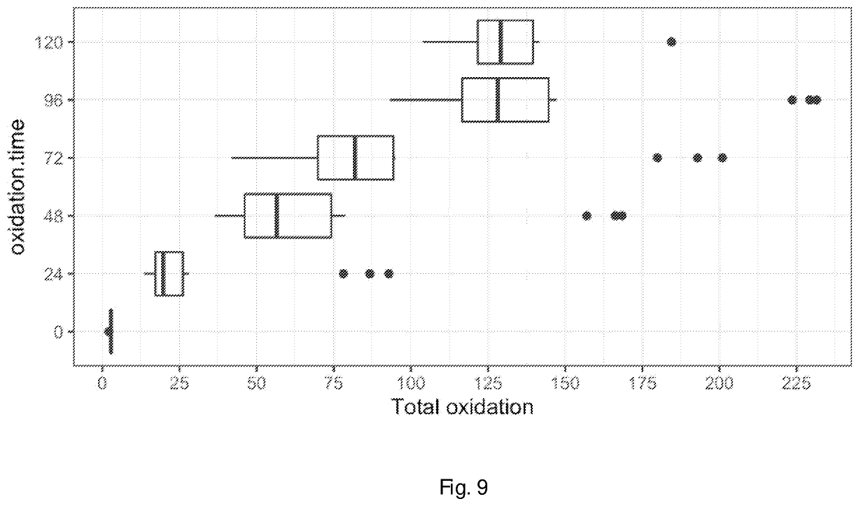

Finally, the inventors estimated the distribution of the diffusion coefficients and total oxidation values associated with different oxidation treatments, where treatment time varied from 0 to 120 hours (FIGS. 8 and 9, respectively).

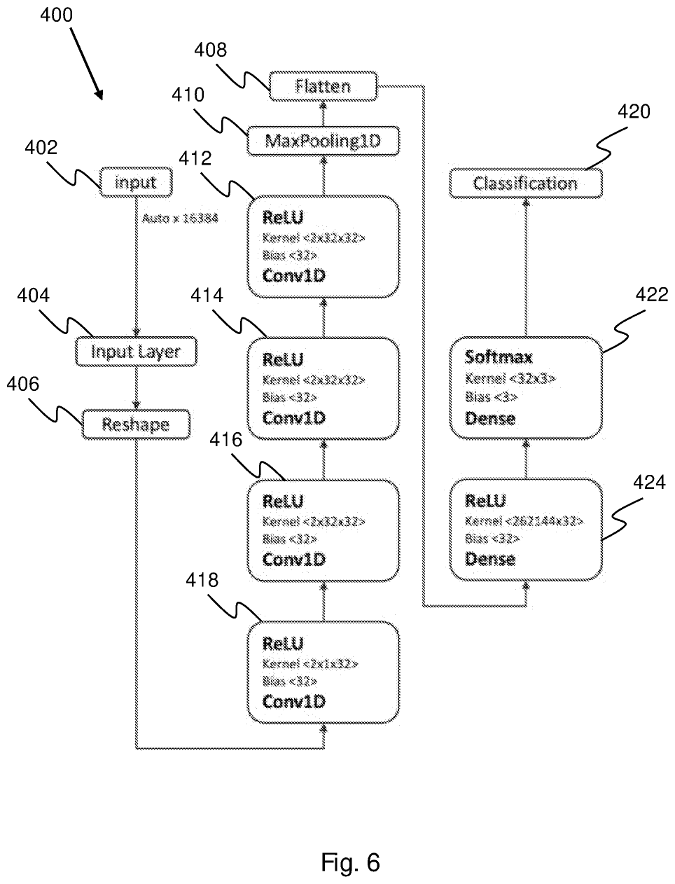

FIG. 6 shows an example of a tailor-made Convolutional Neural Network (CNN) architecture consisting of several key components: an input layer (402, 404, 406), four convolution layers (412, 414, 416, 418), a pooling layer (410, 408, and two dense layers (424, 422). The input layer receives the data (Raw T2 relaxation curves) that is to be processed by the network. The four convolution layers identify and extract features from the input data. The pooling layer then reduces the spatial dimensions of the feature maps generated by the convolution layers. Finally, the two dense layers serve as the classifier (420), determining the output class label based on the processed data.

More specifically:

Input Layer: The input layer (402, 404, 406) contains the raw NMR acquired signal, one for each different acquisition. The acquisition consists of a single n-dimensional vector (n=16384). Having several hundred training vectors varying according to the training session, the m dimension of the input was set on: Auto, thus, changing according to necessity.

Reshape layer: Reshape is a flexible operation that can be used in various ways in CNNs. In the present case, it is used to keep the input (a.k.a. tensor) to the same flat (1 dimensional) shape suitable for the defined task.

Conv1D Layers: The Conv1D layers (412, 414, 416, 418) form together a 1-dimensional convolutional layer of the CNN. It is sometimes called the feature extractor layer because the signal's features are extracted within this layer. The input signal is connected to the Conv1D layer to perform convolution operation, that is, calculating the dot product between the receptive field (it is a local region of the input image that has the same size as that of the filter) and the filter. The result of the operation is a single integer that contributes to forming the total output volume. Then we slide the filter over the next receptive field of the same input signal by a Stride and do the same operation again. The same process is repeated until we go through the whole signal. The output is the input for the next Conv1Dlayer, namely, four layers in total.

ReLU: Conv1D also contains a ReLU activation making all negative values zero. ReLU (Rectified Linear Unit) is an activation function used in Convolutional Neural Networks (CNNs). ReLU takes a real-valued input and returns the maximum of that input and 0. In mathematical terms, the ReLU function is defined as:

f(x)=max(0,x)

where x is the input to the function.

ReLU has several advantages over other activation functions. First, it is computationally efficient to compute, since it involves only simple element-wise operations. Second, ReLU can help addressing the problem of vanishing gradients that can occur in deep networks, by ensuring that gradients can still flow through the network even for large input values. Finally, ReLU has been shown to work well in practice for a wide range of tasks, including 1-dimensional signals tasks.

To summarize, ReLU is a popular activation function in CNNs that helps introducing non-linearity into the network, while also providing computational efficiency and help in addressing the problem of vanishing gradients.

Kernel: The kernel is a small matrix of weights that is used to perform convolutional operations in the convolutional layer; 2×1×32 is the dimension of the kernel that was chosen for this CNN. The size of the kernel (2×1×32) is much smaller than the input data (1×16384), allowing it to capture local patterns and features in the input. The kernel slides across the input data performing element-wise multiplication at each position and then summing up the results to produce a single output value. This process repeats for each position in the input data, producing a new output tensor representing the input's filtered version. The kernel weights are learned during training using back-propagation, allowing the network to adapt to the specific task at hand. The Conv1D, i.e. the convolutional layer, has multiple (i.e. 2) kernels, each of which learns to capture different features or patterns in the input data. In summary, the kernel of a convolutional layer is a small matrix of weights used to perform convolutional operations on the input data, allowing the network to learn local patterns and features useful for the given task.

Bias: All the layers have a 32-dimensional bias term, one for each filter in the layer. A convolutional layer in a 1D CNN applies a set of filters to the input data and produces a set of feature maps as output. Each filter is a set of learnable weights applied to a small input data window at a time to detect certain patterns or features. The bias term is a learnable scalar value added to the output of each filter, allowing the model to shift the output of the filter up or down. The convolutional layer has 32 filters with a kernel size of 2 and a ReLU activation function. The use_bias parameter is set to True, meaning bias terms will be included in the layer.

MaxPoolingID layer: MaxPoolingID 410 is a type of layer used for one-dimensional data. It reduces the dimensionality of the input by sliding a window of fixed size over the input and taking the maximum value within that window. More specifically, MaxPoolingID performs a downsampling operation on the input along the temporal dimension (i.e., along the length of the input sequence). The operation takes a window of size pool_size=2 and slides it across the input, taking the maximum value within the window for each feature map. The output is a downsampled version of the input, where the length of the sequence is reduced by a factor of pool_size.

Flatten layer: The Flatten layer 408 converts the output of the convolutional layers, a 3D tensor, into a 1D tensor to pass it to a fully connected layer. The Flatten layer essentially flattens the 2D tensor output from the last convolutional layer back into a 1D tensor, where each element in the 1D tensor corresponds to a unique feature. The output shape of the Flatten layer is determined by the number of filters and the dimensions of the filters in the last convolutional layer. The flattened tensor is then passed to a fully connected layer with 32 units and a ReLU activation function.

Dense layers: A dense (i.e., fully connected) layer is a simple layer of 32 neurons in which each neuron receives input from all the neurons of the previous layer, thus called as dense. Dense layer is used to classify the features obtained as output from convolutional layers. Each of the two dense layers contains 32 of such neurons. This involves weights and the corresponding 32 biases. It connects neurons in one layer to neurons in another layer. Each Layer in the Neural Network contains neurons, which compute the weighted average of its input plus the biases. This weighted average is passed through a non-linear function called the activation function. In the case of a Dense layer with input shape (batch_size, 262144) and 32 units, the kernel will have dimensions (262144, 32). This means that there are 262144 weights connecting each of the 262144 input features to each of the 32 units in the Dense layer. The weights in the kernel matrix are learned during training using back-propagation and gradient descent. The values of the weights will change during training to minimize the loss function and improve the model's accuracy on the task. The first activation function is a ReLU (described above) the second is a Softmax described below. The dense layers are used to classify the features into different categories by training.

Softmax: Softmax 422 is the last layer of the CNN. It resides at the end of the fully connected network. Softmax is designed for multi-class classification, i.e., classification into three different possible outputs representing three different oil oxidation qualities.

Classification: The multi-class (3 classes) classification layer is the neural network's final layer that produces the model output as a probability distribution over the three possible classes (in this specific case). The classification layer is implemented as a fully connected (Dense) layer with three units, one for each class in the classification task. The activation function used in the classification layer is a softmax function, which ensures that the output values are between 0 and 1 and sum to 1, making it possible to interpret the output as probabilities. The classification layer is added as a Dense layer with three units and a softmax activation function. This is appropriate for a classification task with three possible classes. During training, the model learns to assign higher probabilities to the correct class labels and lower probabilities to the incorrect ones. The predicted class for each input will be the class with the highest probability in the output.

FIG. 8 provides test results showing the accuracy of diffusion coefficients as an estimator of the oxidative treatment imposed on the oil samples, where treatment varied from 0 to 120 hours. Results show that diffusion coefficients decrease when the oxidative treatment increase (on the y-axis: oxidation time). The y-axis indicates the oxidation treatment's duration (in hrs.), and the x-axis indicates the associated diffusion measurements.

FIG. 9 provides test results showing the accuracy of total oxidation as an estimator of the oxidative treatment imposed on the oil samples, where treatment varied from 0 to 120 hours. Results show that the diffusion coefficients decrease when the oxidative treatment increase (on the y-axis: oxidation time). The y-axis indicates the duration (in hrs.) of the oxidation treatment, and the x-axis indicates the associated measurements of total oxidation. On each box, the central mark indicates the median, and the bottom and top edges of the box indicate the 25^{th} and 75^{th} percentiles, respectively. The lines extend to the most extreme data points not considered outliers, and the outliers are plotted individually using the ‘dot’ marker symbol.

Annex 1-an example showing a subset of the available
database

| | exp. | exp. | Dif- | Per- | Anisi- | Total- |
|---|---|---|---|---|---|---|
| Experiment | oxidation | repe- | fusion- | oxide- | dine- | oxi- |
| date | time | tition | coeff. | val. | val. | dation |
| 14 Nov. 2021 | 0 | 1 | 0.03 | 3.02 | 1.54 | 3.08 |
| 14 Nov. 2021 | 0 | 10 | 0.04 | 2.68 | 1.54 | 2.77 |
| 14 Nov. 2021 | 0 | 2 | 0.05 | 2.62 | 1.54 | 2.71 |
| 14 Nov. 2021 | 0 | 3 | 0.04 | 2.77 | 1.54 | 2.86 |
| 14 Nov. 2021 | 0 | 4 | 0.04 | 2.24 | 1.54 | 2.32 |
| 14 Nov. 2021 | 0 | 5 | 0.04 | 2.88 | 1.54 | 2.96 |
| 14 Nov. 2021 | 0 | 6 | 0.03 | 3.28 | 1.54 | 3.35 |
| 14 Nov. 2021 | 0 | 7 | 0.04 | 2.68 | 1.54 | 2.75 |
| 14 Nov. 2021 | 0 | 8 | 0.04 | 3.18 | 1.54 | 3.25 |
| 14 Nov. 2021 | 0 | 9 | 0.04 | 2.65 | 1.54 | 2.73 |
| 14 Nov. 2021 | 120 | 1 | 0.02 | 184.42 | 125.05 | 184.46 |
| 14 Nov. 2021 | 120 | 2 | 0.02 | 141.63 | 125.05 | 141.67 |
| 14 Nov. 2021 | 120 | 3 | 0.02 | 119.07 | 125.05 | 119.10 |
| 14 Nov. 2021 | 24 | 1 | 0.04 | 19.39 | 13.99 | 19.47 |
| 14 Nov. 2021 | 24 | 2 | 0.04 | 27.90 | 13.99 | 27.98 |
| 14 Nov. 2021 | 24 | 3 | 0.04 | 25.32 | 13.99 | 25.40 |
| 14 Nov. 2021 | 48 | 1 | 0.04 | 78.61 | 92.85 | 78.69 |
| 14 Nov. 2021 | 48 | 2 | 0.03 | 72.59 | 92.85 | 72.66 |
| 14 Nov. 2021 | 48 | 3 | 0.03 | 59.76 | 92.85 | 59.83 |
| 14 Nov. 2021 | 72 | 1 | 0.03 | 69.22 | 110.14 | 69.28 |
| 14 Nov. 2021 | 72 | 2 | 0.03 | 84.98 | 110.14 | 85.04 |
| 14 Nov. 2021 | 72 | 3 | 0.02 | 60.62 | 110.14 | 60.67 |
| 14 Nov. 2021 | 96 | 1 | 0.03 | 147.27 | 120.84 | 147.33 |
| 14 Nov. 2021 | 96 | 2 | 0.03 | 143.65 | 120.84 | 143.70 |
| 14 Nov. 2021 | 96 | 3 | 0.02 | 126.92 | 120.84 | 126.97 |
| 21 Nov. 2021 | 0 | 1 | 0.04 | 3.02 | 0.64 | 3.10 |
| 21 Nov. 2021 | 0 | 10 | 0.04 | 2.68 | 0.64 | 2.77 |
| 21 Nov. 2021 | 0 | 2 | 0.04 | 2.62 | 0.64 | 2.70 |
| 21 Nov. 2021 | 0 | 3 | 0.04 | 2.77 | 0.64 | 2.85 |
| 21 Nov. 2021 | 0 | 4 | 0.04 | 2.24 | 0.64 | 2.32 |
| 21 Nov. 2021 | 0 | 5 | 0.04 | 2.88 | 0.64 | 2.96 |
| 21 Nov. 2021 | 0 | 6 | 0.04 | 3.28 | 0.64 | 3.37 |
| 21 Nov. 2021 | 0 | 7 | 0.04 | 2.68 | 0.64 | 2.76 |
| 21 Nov. 2021 | 0 | 8 | 0.04 | 3.18 | 0.64 | 3.26 |
| 21 Nov. 2021 | 0 | 9 | 0.05 | 2.65 | 0.64 | 2.74 |
| 21 Nov. 2021 | 120 | 1 | 0.03 | 104.71 | 88.04 | 104.77 |
| 21 Nov. 2021 | 120 | 10 | 0.03 | 138.68 | 88.04 | 138.74 |
| 21 Nov. 2021 | 120 | 2 | 0.03 | 121.72 | 88.04 | 121.77 |
| 21 Nov. 2021 | 120 | 3 | 0.03 | 103.83 | 88.04 | 103.89 |
| 21 Nov. 2021 | 120 | 4 | 0.03 | 125.18 | 88.04 | 125.23 |
| 21 Nov. 2021 | 120 | 5 | 0.03 | 141.11 | 88.04 | 141.17 |
| 21 Nov. 2021 | 120 | 6 | 0.03 | 129.02 | 88.04 | 129.08 |
| 21 Nov. 2021 | 120 | 7 | 0.03 | 121.64 | 88.04 | 121.69 |
| 21 Nov. 2021 | 120 | 8 | 0.02 | 139.50 | 88.04 | 139.55 |
| 21 Nov. 2021 | 120 | 9 | 0.03 | 133.39 | 88.04 | 133.45 |
| 21 Nov. 2021 | 24 | 1 | 0.04 | 20.54 | 10.66 | 20.62 |
| 21 Nov. 2021 | 24 | 10 | 0.04 | 16.13 | 10.66 | 16.22 |
| 21 Nov. 2021 | 24 | 2 | 0.04 | 13.39 | 10.66 | 13.47 |
| 21 Nov. 2021 | 24 | 3 | 0.04 | 19.87 | 10.66 | 19.94 |
| 21 Nov. 2021 | 24 | 4 | 0.04 | 16.87 | 10.66 | 16.95 |
| 21 Nov. 2021 | 24 | 5 | 0.04 | 16.35 | 10.66 | 16.42 |
| 21 Nov. 2021 | 24 | 6 | 0.04 | 22.70 | 10.66 | 22.79 |
| 21 Nov. 2021 | 24 | 7 | 0.04 | 17.22 | 10.66 | 17.30 |
| 21 Nov. 2021 | 24 | 8 | 0.04 | 18.94 | 10.66 | 19.01 |
| 21 Nov. 2021 | 24 | 9 | 0.04 | 18.48 | 10.66 | 18.56 |
| 21 Nov. 2021 | 48 | 1 | 0.04 | 58.77 | 50.56 | 58.84 |
| 21 Nov. 2021 | 48 | 10 | 0.03 | 46.80 | 50.56 | 46.86 |
| 21 Nov. 2021 | 48 | 2 | 0.04 | 56.44 | 50.56 | 56.52 |
| 21 Nov. 2021 | 48 | 3 | 0.04 | 50.00 | 50.56 | 50.08 |
| 21 Nov. 2021 | 48 | 4 | 0.04 | 43.74 | 50.56 | 43.81 |
| 21 Nov. 2021 | 48 | 5 | 0.03 | 36.32 | 50.56 | 36.38 |
| 21 Nov. 2021 | 48 | 6 | 0.03 | 40.23 | 50.56 | 40.29 |
| 21 Nov. 2021 | 48 | 7 | 0.03 | 56.38 | 50.56 | 56.44 |
| 21 Nov. 2021 | 48 | 8 | 0.04 | 54.77 | 50.56 | 54.86 |
| 21 Nov. 2021 | 48 | 9 | 0.04 | 42.48 | 50.56 | 42.55 |
| 21 Nov. 2021 | 72 | 1 | 0.03 | 75.00 | 85.72 | 75.07 |
| 21 Nov. 2021 | 72 | 1C | 0.04 | 93.62 | 85.72 | 93.69 |
| 21 Nov. 2021 | 72 | 3 | 0.03 | 51.94 | 85.72 | 52.00 |
| 21 Nov. 2021 | 72 | 4 | 0.04 | 70.00 | 85.72 | 70.07 |
| 21 Nov. 2021 | 72 | 5 | 0.04 | 75.84 | 85.72 | 75.91 |
| 21 Nov. 2021 | 72 | 6 | 0.03 | 78.57 | 85.72 | 78.64 |
| 21 Nov. 2021 | 72 | 7 | 0.03 | 85.98 | 85.72 | 86.05 |
| 21 Nov. 2021 | 72 | 8 | 0.04 | 94.03 | 85.72 | 94.11 |
| 21 Nov. 2021 | 96 | 1 | 0.03 | 109.11 | 86.83 | 109.17 |
| 21 Nov. 2021 | 96 | 10 | 0.03 | 132.64 | 86.83 | 132.70 |
| 21 Nov. 2021 | 96 | 2 | 0.03 | 119.17 | 86.83 | 119.23 |
| 21 Nov. 2021 | 96 | 3 | 0.03 | 129.16 | 86.83 | 129.23 |
| 21 Nov. 2021 | 96 | 4 | 0.03 | 112.84 | 86.83 | 112.91 |
| 21 Nov. 2021 | 96 | 5 | 0.03 | 93.16 | 86.83 | 93.22 |
| 21 Nov. 2021 | 96 | 6 | 0.03 | 118.91 | 86.83 | 118.97 |
| 21 Nov. 2021 | 96 | 7 | 0.04 | 96.77 | 86.83 | 96.85 |
| 21 Nov. 2021 | 96 | 8 | 0.03 | 134.00 | 86.83 | 134.06 |
| 21 Nov. 2021 | 96 | 9 | 0.03 | 117.80 | 86.83 | 117.86 |
| 28 Nov. 2021 | 0 | 1 | 0.04 | 1.99 | 1.67 | 2.06 |
| 28 Nov. 2021 | 0 | 3 | 0.04 | 2.22 | 1.67 | 2.31 |
| 28 Nov. 2021 | 24 | 1 | 0.04 | 92.77 | 34.41 | 92.85 |
| 28 Nov. 2021 | 24 | 2 | 0.04 | 86.55 | 34.41 | 86.63 |
| 28 Nov. 2021 | 24 | 3 | 0.04 | 78.02 | 34.41 | 78.10 |
| 28 Nov. 2021 | 48 | 2 | 0.03 | 168.32 | 73.78 | 168.38 |
| 28 Nov. 2021 | 48 | 3 | 0.03 | 166.20 | 73.78 | 166.26 |
| 28 Nov. 2021 | 72 | 1 | 0.03 | 179.87 | 93.16 | 179.92 |
| 28 Nov. 2021 | 72 | 2 | 0.02 | 200.90 | 93.16 | 200.95 |
| 28 Nov. 2021 | 72 | 3 | 0.03 | 192.84 | 93.16 | 192.89 |
| 28 Nov. 2021 | 96 | 1 | 0.02 | 231.45 | 113.78 | 231.48 |
| 28 Nov. 2021 | 96 | 2 | 0.02 | 223.56 | 113.78 | 223.59 |
| 28 Nov. 2021 | 96 | 3 | 0.01 | 229.25 | 113.78 | 229.27 |

Annex 2 - Descriptive statistics of selected variables

| Diffusion-coeff. | Peroxide-val. | Anisidine-val. | Total-oxidation |
|---|---|---|---|
| Min.: 0.01224 | Min.: 1.99 | Min.: 0.64 | Min.: 2.064 |
| 1st Qu.: 0.0290⁰ | 1st Qu.: 16.3⁰ | 1st Qu.: 10.6⁶ | 1st Qu.: 16.373 |
| Median: 0.03450 | Median: 64.92 | Median: 62.17 | Median: 64.972 |
| Mean: 0.03392 | Mean: 72.76 | Mean: 55.54 | Mean: 72.829 |
| 3rd Qu.: 0.0400⁰ | 3rd Qu.: 119.7⁹ | 3rd Qu.: 88.0⁴ | 3rd Qu.: 119.847 |
| Max.: 0.04700 | Max.: 231.45 | Max.: 125.05 | Max.: 231.483 |

### Example 2

### A Synthetic Representation of the T₂-Experiment Database and its Correlation to Different Oxidation Levels

To initiate a time domain (TD) NMR ¹H relaxation experiment, a spin magnetization was created in a low-field NMR by a homogeneous magnetic field. Then, a sensor (i.e., antenna) measured how the initial state changed over time, i.e., the T₂ relaxation. This is a complex phenomenon, but at its most fundamental level, it is a decoherence of the initial nuclear spin-spin magnetization on the transverse (x/y) plane. A so-called T₂ signature is a collection of T₂ time constants for a given material. In this specific case, the inventors run experiments on a single type of oil found in foods, for example, linseed, at different thermal stimulated oxidation levels.

T₂ relaxation fingerprints/signatures resulting from NMR experiments on linseed oils exposed to various oxidative treatments for 0, 24, 48, 72, and 120 hrs, as described in previously published papers (Resende et al., 2021 and Osheter et al., 2022). Each T₂ relaxation signature is the computed summary of multiple experimental repetitions for the same treatment, thus representing the average profile for a given oxidation treatment. The synthetic computed signature results from the summary of complicated algebraic inverse Laplace transformation (ILT) (as described in detail in a previous patent application, PCT/IL2018/050279) of various experimental repetitions and show a similar typical shift depending on the oxidation level. However, this procedure based on the ILT consumes relatively much operation time. Therefore, in the present application, a new approach based on using T₂ rapid collection of raw relaxation signals data was developed and used. FIG. 8 shows the raw signals (i.e., exponential decay curves) resulting from the same NMR relaxation experiments. More specifically, raw exponential decay curves of ¹H NMR T₂ relaxation signals of linseed oil collected by the CPMG method from LF NMR after different thermal oxidation levels/times. Each line in the figure corresponds to a unique T₂ relaxation time signal obtained from a linseed oil sample with a different level of oxidation (0 hours, 12 hours, 24 hours, 48 hours, 96 hours, and 120 hours). The plot illustrates the effect of thermal oxidation on the T₂ relaxation time of the linseed oil samples. Based on a matrix of a huge number (16,000) of relaxation testing points, these obtained relaxation curves are difficult to differentiate by human eyes; however, all the exact relaxation curves are easily recognized by the computer. This approach opens the way to develop a rapid and high efficient machine learning technology, as described below.

### Example 3: Correlation of Predicted Results and Experimental Results

Table 3 shows the correlation of predicted results with target measurements on real experimental results. More specifically, the inventors used an artificial neural network to predict oxidation time, below 48 hours versus above 48 hours and up to 120 hours. The observations were dichotomized to utilize the NMR signatures to distinguish between low and high oxidation times. The observations were divided into two groups, one used for training the ANN (80% of the samples) and the other group to test the prediction accuracy on samples never used for training (20% of the sample). The procedure was repeated 100 times using a k-fold cross-validation method (where k was 100). Preliminary results were found encouraging. The ANN recognized the oxidation time with an accuracy of 83.5% CI95: (0.8206, 0.8494). Overall, ANN sensitivity and specificity were above 0.75, confirming satisfactory preliminary results.

TABLE 3
Sensitivity-Specificity Matrix and Statistics

Reference

| 1 | 0 | Prediction |
|---|---|---|
| 256 | 1344 | 0 |
| 828 | 172 | 1 |

Accuracy: 0.8354;
95% CI: (0.8494, 0.8206);
No Information Rate: 0.5831;
P-Value [Acc > NIR]: <2.2e−16;
Kappa: 0.6576;
Mcnemar's Test P-Value: 6.022e−05;
Sensitivity: 0.8865;
Specificity: 0.7638;
Pos. Pred. Value: 0.8400;
Neg. Pred. Value: 0.8280;
Prevalence: 0.5831;
Detection Rate: 0.5169;
Detection Prevalence: 0.6154;
Balanced Accuracy: 0.8252.

### Example 4: LF ¹H NMR Determination of Average Self-Diffusion D Values for Estimation of Oxidation

A rapid procedure for oil product development potentially needed for food products is described below using LF ¹H NMR for calculating self-diffusion values within minutes for direct online analysis. TD NMR determination of self-diffusion (SD or D) values of Saturated, Monounsaturated, and Polyunsaturated oils TD NMR is well accepted for characterizing the chemical and physical status of foods with fatty acids and esters. In comparison, conventional determination using PV or PAV or TOXOX, which takes hours of extracted samples for each value, demonstrates the efficacy of TD LF ¹H NMR in characterizing the degree of oxidation and the molecular structure (peroxide, aldehyde, polymers) of the oxidized products as an online analytical method in food production. For example, in Table 4 below, there is a correlation between self-diffusion values (SD) of linseed oil and one of the three parameters analyzed by common conventional tests of PV, PAV, and TOTOX using the same LSO samples during the entire period of thermal oxidation (120 h). It should be noted that the best correlation was found between SD and PAV, suggesting a better relationship correlating proton mobility/movement within the linseed oil and aldehydes formation that represents the oil chemical-structural changes during the initial stages of oxidation.

TABLE 4
Self-diffusion (D) values of Saturated
Monounsaturated and Polyunsaturated edible oils during
thermal oxidation stimulation conditions at 80º C.

| | | | | | | |
|---|---|---|---|---|---|---|
| Time of heating (hr) | 0 | 24 | 48 | 72 | 96 | 120 |

Saturated:

| | | | | | | |
|---|---|---|---|---|---|---|
| Butter | 0.161* | 0.026 | 0.033 | 0.029 | 0.033 | 0.029 |
| Coconut oil | 0.037 | 0.038 | 0.036 | 0.036 | 0.035 | 0.037 |

Mono-Unsat :

| | | | | | | |
|---|---|---|---|---|---|---|
| Olive oil | 0.03 | 0.028 | 0.025 | 0.024 | 0.024 | 0.026 |
| Canola oil | 0.03 | 0.027 | 0.028 | 0.023 | 0.019 | 0.019 |

Poly-Unsat:

| | | | | | | |
|---|---|---|---|---|---|---|
| Soy oil | 0.034 | 0.029 | 0.028 | 0.022 | 0.018 | 0.012 |
| Linseed oil | 0.04 | 0.041 | 0.034 | 0.03 | 0.024 | 0.018 |

The suitability of using D values in Table 4 for monitoring the oxidation status of oils is shown by the relationship between para-anisidine values (PAV) of aldehyde concentration and diffusion coefficient (D) of various edible oils during all the same times of thermal oxidation at 80° C. A good correlation is obtained for the self-diffusion of the highly oxidized oils. Correlation between Linseed oil (LSO) diffusivity (D) with PV; PAV; TOTOX (scattering graphs/PCA) values. In this patent application, online analysis of the extent of oxidation is not available by current methods such as PV and PAV. The inventors overcome this limitation with an NMR sensor that rapidly and accurately determines diffusion (D) or equivalently self-diffusion (SD) value. This is based on an analysis of the sample's self-diffusions (D) values being determined by spin-spin time values of the different alkyl chain protons (¹H). D correlates well to PAV values of, for example, aldehydes reflecting chemical and morphological changes of samples during oxidation and is an excellent marker of the oxidation status of the samples. Thus the present invention of performing D analysis by LF ¹H NMR indicates that conventional methodologies, such as PV for peroxides and PAV for aldehydes and total oxidation by TOTOX used for determining food oxidation with FA oil foods, can be substituted by a much faster determination of the foods oxidative status based on LF ¹H NMR determination of average self-diffusion D values.

### Example 5: Convolutional Neural Network (CNN) Organization and Training

A convolutional Neural Network (CNN) is an ANN AI system. The CNN training includes: (a) Inducing oxidation in the sample; (b) Activating the NMR to acquire a T₂ signal; and (c) training and testing the CNN. In particular: each oil sample (1 in FIG. 4) was treated with one of six different thermal treatments to induce different levels of oxidation on food-grade, pure, high-quality linseed oil (LSO). The treatment duration varied from a 0 hour (namely, no induced oxidation) to a maximum of 120 hours, where the oxidation was expected to be at its highest level. The different treatments were denoted as: 0 hr, 12 hr, 24 hr, 48 hr, 96 hr, 120 hr). Subsequently (2 in FIG. 4), each sample was analyzed with: (i) LF-NMR, to acquire raw the T₂ relaxation curves; and ii) LF-NMR gradient pulse analysis of self-diffusion coefficient D and conventional standard chemical lab methods (PV, p-AV and TOTOX was calculated). The oxidation measurements were converted into ordinal classes as follow: Good—non-oxidized oil, Fair—partially oxidized oil and Bad—highly oxidized oil according to the classification criteria. These are the ground-truth for the oxidation levels achieved with the treatments in the previous step. The LF-NMR raw T₂ relaxation signal acquisitions were not transformed into the frequency domain, allowing for the fast-collected raw signals to be used as such. By binding these measurements (3 in FIG. 4) together, a basic database of labeled T₂ signals was formed. Subsequently, (4 in FIG. 4), several convolutional neural networks (CNN) were trained, benchmarked, and fine-tuned for the classification task of the T₂ signal into three classes corresponding to three different oxidation levels (bad, fair, and good). The CNN included: i) encoding modules used for automatic feature extraction and data dimensionality reduction; and ii) a decoding module, namely a supervised network of liner combiners (as well as the corresponding activation functions) that was trained by a gradient descent algorithm (i.e. ADAM, the Adaptive Moment Estimation method. The convolutional layers of the CNN included a set of filters for the input data to determine local features, such as edges and shapes. The convolutional layers applied a set of filters to the input data, designed to extract essential features from the data. The pooling layers reduced the dimensionality of the data by summarizing the output of the convolutional layers over a local region. The output of the CNN was then passed through one or more fully connected layers, which in turn applied weights to the output of the convolutional layers, to predict or decide. Finally, one of the three predefined possible oxidation classes was indicated by the deep convolution neural network (DCNN) (FIG. 2 step 205). Different CNN configurations were benchmarked to improve the prediction accuracy. The following results focus only on the final optimized configuration.

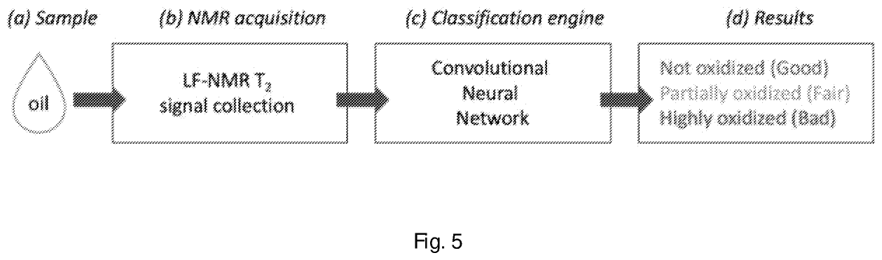

FIG. 5 schematically summarizes the system's setup and the typical workflow for analyzing oil samples using a low field Nuclear Magnetic Resonance (LF-NMR) and a Convolutional Neural Network (CNN). In the first step (a), a drop of oil was scanned using the LF-NMR machine to obtain its T₂ signal, which indicates the oil's relaxation time. In the next step (b), the obtained T₂ signal was used as an input to the CNN. Then, the CNN (c) processed the T₂ signal, indicating (d) the degree (class) of oxidation the oil sample has undergone.

By combining the conventional standard chemical methods and self-diffusion coefficient D, it is possible to create a broad profile for LSO samples and their oxidation. Since PV and p-AV were found to correlate with the coefficient D (that, in turn, correlated to the initial and later stages of oxidation, respectively), the PV values were expected to increase and afterward decrease. The PV and D values were used to categorize the LSO into 3 groups, as in Table 5 below. Cutoff values for PV are 30 mmol/kg and for D are 0.03*10−9 m2/s for non-oxidized, ‘Good’ LSO. Cutoff range of 30-50 mmol/kg of PV and D range of 0.03−0.02*10−9 m2/s for partially oxidized, ‘Fair’ LSO. Cutoff of PV higher than 50 mmol/kg and D values lower than 0.02*10−9 m2/s for highly oxidized, ‘Bad’ LSO. With those criteria, 126 ‘Good’ samples, 77 ‘Fair’ samples, and 187 Bad’ samples were determined.

TABLE 5
Criteria for dividing oil samples into the following
three categories: Good, Fair, and Bad.

| | D¹ range | PV² range | |
|---|---|---|---|
| Catergory | (*10−9 m2/s) | (mmol/kg) | Total samples |
| ‘Good’ | >0.03 | <20 | 126 |
| ‘Fair’ | 0.02-0.03 | 20-50 | 77 |
| ‘Bad’ | ≤0.02 | ≥50 | 187 |

¹Diffusion coefficient
²Peroxide value

### Example 6: Optimimized Classification Performances

The performance of the CNN over the testing set is shown in Tables 6 and 7. The metrics evaluate the ability of the CNN to measure the oxidation level in food materials and chemicals. The F1 score, a measure of the model's accuracy on unseen test data, was calculated as the average of precision and recall. It is a measure of the CNN's performance. The F1 score is defined as follows:

$$F 1 - score = 2 * ( precision * recall ) / ( precision + recall )$$

“Precision” is the proportion of true positive predictions made by the model. “recall” is the proportion of positive instances correctly identified by the model. The term “support” refers to the number of samples in the test set that belong to a particular class.

The CNN achieved state-of-the-art results over a wide range of different samples at different oxidation levels, resulting in approximately 99% percent of overall accuracy on Very Bad classes, approximately 77% overall accuracy on Fair classes, and approximately 94% on Good classes. The false positive and false negative rates were low or extremely low, ranging from 1% to 6%, depending on the case. The weighted average F1 score was approximately 92%, comparable with state-of-the-art computerized pattern recognition performances. These results prove the efficacy of rapidly measuring the oxidation level of chemicals and products, beyond what is currently available. Applicant believes that the results are particularly applicable, but not limited to, materials and chemicals with polyunsaturated fatty acids (PUFA).

FIGS. 8 and 9 show various estimators of classification accuracy for a best-performing CNN obtained during testing and validation.

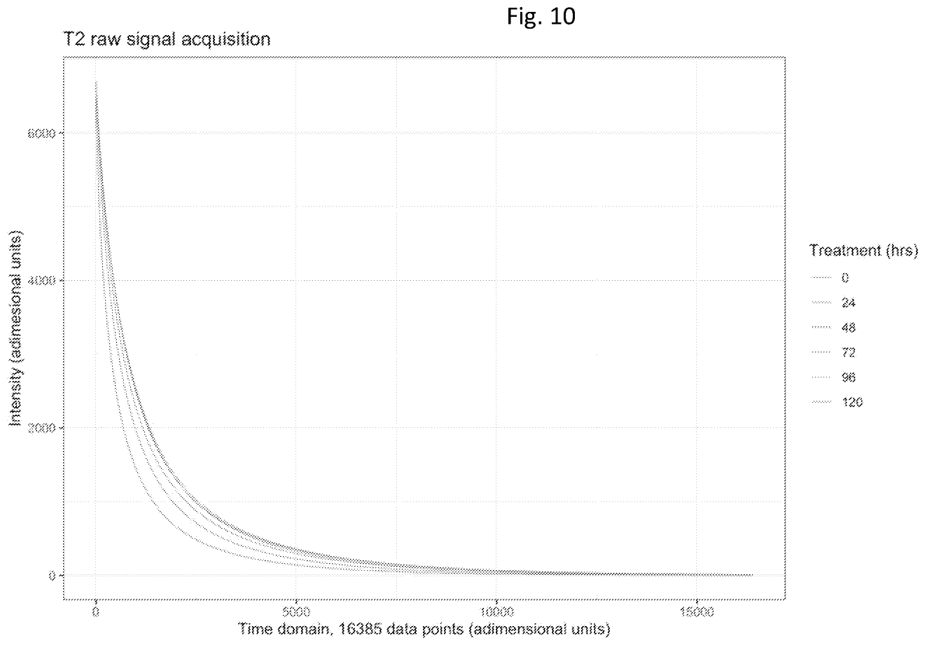

FIG. 10 shows raw exponential decay curves of 1H NMR T2 relaxation signals of linseed oil collected by CPMG method from LF NMR after different thermal oxidation levels/times. Each line corresponds to a unique T2 relaxation time signal obtained from a linseed oil sample with a different level of oxidation (0 hours, 12 hours, 24 hours, 48 hours, 96 hours, and 120 hours). The plot illustrates the effect of thermal oxidation on the T2 relaxation time of the linseed oil samples. The figure shows the mean performances of 30 different CNNs (all with the same architecture) tested on multiple experiments (11,700 tests).

TABLE 6
Confusion matrix for the optimized CNN. The table summarizes the correct
and incorrect predictions made by the classifier and helps to understand
where the model is mistaken. The matrix is constructed by comparing the
predicted class labels to the actual class labels of the test data. The
entries in the matrix show the number of instances that were (a) correctly
classified, (b) incorrectly classified, or (c) not classified at all. Common
metrics such as accuracy, precision, recall, and F1 score can be computed
from the confusion matrix entries to evaluate the classifier's performance.

| | | Predicted condition | | |
|---|---|---|---|---|
| | | Very bad | Fair | Good |
| True | Very bad | True classification | False Positive | False Positive |
| label | | N = 114 (99%) | N = 12 (1%) | N = 0 (0%) |
| | Fair | False Negative | True classification | False Positive |
| | | N = 1 (1%) | N = 65 (77%) | N = 11 (6%) |
| | Good | False Negative | False Negative | True classification |
| | | N = 0 (0%) | N = 7 (6%) | N = 180 (94%) |

TABLE 7
Performances metrics for the optimized CNN

| | Precision | Recall | F1-score | support |
|---|---|---|---|---|
| Very bad | 0.99 | 0.9 | 0.95 | 126 |
| Fair | 0.77 | 0.84 | 0.81 | 77 |
| Good | 0.94 | 0.96 | 0.95 | 187 |

Accuracy

| | | | | |
|---|---|---|---|---|
| Macro average | 0.9 | 0.9 | 0.9 | 390 |
| Weighted average | 0.92 | 0.92 | 0.92 | 390 |

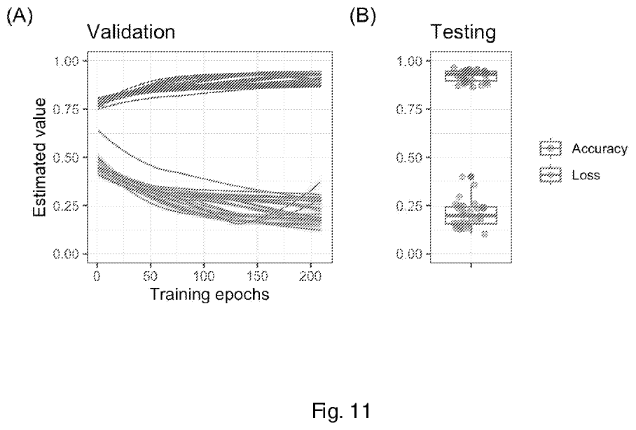

Table 8 and FIG. 11 detail the CNN test performances by oxidation classes.

FIG. 11 illustrates accuracy and loss functions for 30 different Convolutional Neural Networks (CNNs) training sessions. Panel A in the figure displays the evolution of accuracy and loss over time for the validation set, a portion of the data used to monitor the progress of the training process. Typically, as the number of epochs (iterations) increased, the accuracy of the CNN improved, and the loss decreased, indicating that the model learned from the data. Panel B shows the final performance of the CNNs on the testing set, a portion of the data that was not used during training. The data suggest that both the validation and testing performance remain consistent across multiple (n=30) randomly initiated training sessions. This indicates that the CNN was configured correctly, the architecture was appropriate for the data, and the model's performance was reproducible.

In this context, the accuracy indicates how many times (and the respective percentage) the model was correct over the total number of attempts. The precision indicates how well the model predicts a specific output class [true positive/(true positive+false positive)]. Recall indicates how many times the model detected a specific output class [true positive/(true positive+false negative)]. High precision means that the model has made very few false positive predictions and therefore is highly accurate in identifying positive instances. High recall means that the model has identified most of the positive instances and therefore is highly sensitive to the presence of that particular oxidation class. When considering these estimates as a duplet, high precision and high recall indicate a highly accurate model that can detect most instances while minimizing the number of false positives. Conversely, high precision and low recall are less likely to produce false positives. Combining precision and recall in a single index: the “F−1 score” is another measure of the performance on test data. It was calculated as the harmonic mean of the model's precision and recall. The F1 score is defined as:

$$F 1 - score = 2 * ( precision * recall ) / ( precision + recall )$$

The model's performances are summarized in Table 8 below, where the number of repetitions indicates the number of different networks trained independently. At each network reiteration, “support” refers to the number of samples in the test set for a particular class. Thus, each model was tested on 390 samples not used for training. 30 different training sessions were performed (on 30 models that are identical in terms of architecture but are initialized randomly and are tested on different testing sets) for a total of 11,700 tests, where the total number of trials is the product of repetitions (n=30) times support size (n=390). The results indicate that the model achieved comparable state-of-the-art performances over a wide range of different samples at different oxidation levels, with approximately 97% precision for the class “Very Bad”, approximately 88% precision for the “Fair” class, and approximately 94% precision for the “Good” class. The false positives and negatives rates were low or extremely low, ranging from 1% to 6%, depending on the class. Median precision over the entire set was 93% [IQR 87%, 96%]; median recall was 96% [IQR 83%, 98%]. The weighted average F1-score was approximately 0.95 [IQR 0.86, 0.96], comparable with state-of-the-art pattern recognition systems performances.

TABLE 8
Confusion matrix and performances

| | Oxidation class | | | |
|---|---|---|---|---|
| | Bad | Fair | Good | overall |
| Number of | 30 | 30 | 30 | 30 |
| repetitions (n) | | | | |
| Support | 126 | 77 | 187 | 390 |
| (n of samples) | | | | |
| Total number of tests | 3780 | 2310 | 5610 | 11700 |
| Precision (%) | 97% | 88% | 94% | 93% |
| (median [IQR]) | [87%, 0.98%] | [84%, 90%] | [93%, 96%] | [87%, 96%] |
| Recall (%) | 98% | 77% | 97% | 96% |
| (median [IQR]) | [96%, 100%] | [59%, 83%] | [96%, 98%] | [83%, 98%] |
| f1-score | 0.96 | 0.81 | 0.96 | 0.95 |
| (median [IQR]) | [0.91, 0.98] | [0.69, 0.86] | [0.95, 0.97] | [0.86, 0.96] |

This confusion matrix is a performance measurement tool for a classification model. It is used to evaluate the accuracy of a classifier by comparing the predicted values to the actual values in a dataset. The confusion matrix is a table used to evaluate the performance of the proposed CNN classification algorithm, and it summarizes the algorithm's results in a compact form.

The main elements of a confusion matrix are true positive (TP), false positive (FP), true negative (TN), and false negative (FN). The terms are defined as follows:
True positive (TP) is the number of instances correctly classified as positive by the classifier.
False positive (FP) is the number of instances incorrectly classified as positive by the classifier.
True negative (TN) is the number of correctly classified as negative instances by the classifier.
False negative (FN) is the number of instances incorrectly classified as negative by the classifier.

### Interpretation of a Confusion Matrix:

Accuracy: The accuracy of the classifier can be computed using the formula: (TP+TN)/(TP+TN+FP+FN). It gives an overall picture of the correct predictions made by the classifier.
Precision: Precision is the fraction of positive instances that are correctly classified. It can be computed using the TP/(TP+FP) formula. Precision gives an idea of the classifier's ability to avoid false positives.
Recall: Recall is the fraction of positive instances that are correctly classified. Recall gives an idea of the classifier's ability to find all positive instances. It can be computed using the TP/(TP+FN) formula.
F1-score: F1-score is the harmonic mean of precision and recall. It can be computed using the formula: 2*(precision*recall)/(precision+recall). F1-score provides a balanced view of precision and recall.

The matrix was used for Model Selection: to select the best model among several models. The model with the highest accuracy, precision, recall, and F1 score was considered the best model.

Considerations for the Model Improvement: If the classifier has a low recall, it does not find all the positive instances, and the model needs improvement. If the classifier has low precision, it generates too many false positives, and the model needs to be improved. Performance Evaluation: to evaluate the performance of a classifier. It provides a quick and easy way to evaluate a classifier's performance and helps identify the classifier's strengths and weaknesses.

In conclusion, a confusion matrix is a valuable tool for evaluating and improving a classification algorithm. It provides a quick and easy way to evaluate the performance of a classifier, and it helps to identify the strengths and weaknesses of the classifier.

1. A method for determining a level of oxidation in a sample, comprising:
A. a training stage comprising:
a. providing a plurality of food samples;
b. submitting each said food sample to an LF-H¹-NMR device and extracting NMR data for that sample;
c. determining in a lab an oxidation level of each one of said samples;
d. storing in a database for each one of said samples a record reflecting the extracted NMR data and a respective oxidation level;
e. repeating steps a-d for all said plurality of samples;
f. given said plurality of sample records in the database, training and creating a machine-learning unit that, given a sample's NMR data at the unit's input, determines and indicates an oxidation level at the unit's output;
B. a real-time stage comprising:
g. during real-time, extracting real-time NMR data for a food sample;
h. submitting the real-time NMR data to said machine-learning unit; and
i. based on said real-time data, determining by said machine learning unit a respective oxidation level for that sample.

2. The method of claim 1, wherein the sample is a food sample containing oxidation-susceptible components.

3. The method of claim 1, wherein the NMR data is selected from one or more of, NMR T1 energy relaxometry data, NMR T2 relaxometry data, and NMR T1-T2 energy relaxometry data.

4. The method of claim 1, wherein each said record forms labeled data for use at the training stage of the machine learning unit.

5. The method of claim 1, wherein each said oxidation level is reflected by relaxometry and self-diffusion signals acquired from the sample.

6. The method of claim 1, wherein said NMR data comprising exponential decay curves.

7. The method of claim 1, wherein said machine learning training and operation are based on pattern recognition of crude proton energy-time decay curves.

8. The method of claim 2, wherein said real-time stage is performed online during one or more of the food's preparation, storage, transportation, or cooking phases.

9. The method of claim 1, wherein the sample being analyzed for oxidation contains mono or polyunsaturated fatty acids (PUFA), either in solid, liquid, or emulsion combining different phases.

10. A system for determining a level of oxidation in a sample, comprising:
an LF-NMR device configured to extract NMR data from a sample and convey the same into a pre-trained machine-learning unit; and
a pre-trained machine-learning unit configured to receive said NMR data and to determine a level of oxidation within said sample based on said NMR data.

11. The system of claim 10, wherein the sample is a food sample containing oxidation-susceptible components.

12. The system of claim 10, wherein the NMR data is selected from one or more of, NMR T1 relaxometry data, NMR T2 relaxometry data, and NMR T1-T2 relaxometry data.

13. The system of claim 10, wherein each said oxidation level is reflected by relaxometry and self-diffusion signals acquired from the sample.

14. The system of claim 10, wherein the determination of the oxidation level is based on pattern recognition of crude proton energy decay curves.

15. The system of claim 11, configured for online determination of the oxidation level during one or more of the food's preparation, storage, transportation, or cooking phases.
